(19) United States
(12) Patent Application Publication
(10) Pub. No.: US 2019/0385715 A1
(43) Pub. Date: Dec. 19, 2019

(54) SYSTEMS AND METHODS FOR FACILITATING COMPUTER-ASSISTED LINKAGE OF HEALTHCARE RECORDS

(71) Applicant: KONINKLIJKE PHILIPS N.V., EINDHOVEN (NL)

(72) Inventors: Wei Wang, Somerville, MA (US); Reza Sharifi Sedeh, Malden, MA (US); Qingxin Wu, Lexington, MA (US); Yugang Jia, Winchester, MA (US)

(21) Appl. No.: 16/334,232

(22) PCT Filed: Sep. 26, 2017
(86) PCT No.: PCT/IB2017/055819

§ 371 Date: Mar. 18, 2019

Related U.S. Application Data
(60) Provisional application No. 62/401,445, filed on Sep. 29, 2016.

(51) Int. Cl. G16H 10/60; G16H 50/20; G16H 50/30; G16H 50/70

(52) U.S. Cl. CPC G16H 10/60; G16H 50/70; G16H 50/30; G16H 50/20

(57) ABSTRACT

The present disclosure pertains to computer-assisted linkage of healthcare records. In some embodiments, a first portion of a collection of healthcare records of individuals may be processed using a set of record attributes (corresponding to strong identifiers) that includes one or more record attributes corresponding to strong identifiers. Based on such processing, a first set of matches between healthcare records of the first collection portion may be predicted, and a number of matches in the first set of matches may be determined. At least one other portion of the collection of healthcare records of individuals may be processed using another set of record attributes that includes one or more record attributes different from the strong-identifier-corresponding record attributes. Based on the number of matches in the first set of matches, the processing of the other collection portion with respect to predicting healthcare record matches may be caused to stop.

### BACKGROUND

### 1. Field

The present disclosure relates to systems and methods for facilitating computer-assisted linkage of healthcare records.

### 2. Description of the Related Art

Computer-assisted data linkage systems are generally used to facilitate matching and linking of data by automating one or more operations to match and link data. Typical data linkage systems, however, waste significant amounts of computational resources (e.g., processing resources, memory resources, network bandwidth, etc.) continuing to process a collection of records for matches, for example, when few undetermined matches (relative to the amount of matches already found) remain. Although an arbitrary predefined threshold may be strictly enforced to stop processing to limit computational resource waste, the strict use of an arbitrary predefined threshold often results in insufficient matches. Specifically, for example, while the strict use of a particular predefined threshold for processing a first collection of records may produce sufficiently complete matching, it is very likely that processing of another collection of records using the same predefined threshold will produce insufficiently complete matching, where the other record collection has record inconsistencies different from those in the first record collection, record attributes different from those in the first record collection, or other differences.

### SUMMARY

Accordingly, one aspect of the disclosure relates to a system configured for facilitating computer-assisted linkage of healthcare records using strong identifiers. The system comprises one or more hardware processors configured by machine-readable instructions to process, using a set of record attributes corresponding to strong identifiers, a first portion of a collection of healthcare records of individuals to predict which healthcare records of the first collection portion have matching values with respect to the set of record attributes. The strong-identifier-corresponding set of record attributes includes one or more record attributes corresponding to strong identifiers. The prediction indicates a first set of matches between healthcare records of the first collection portion. A number of matches in the first set of matches is determined. Using another set of record attributes, processing is performed on at least one other portion of the collection of healthcare records of individuals. This is done to predict which healthcare records of the other collection portion have matching values with respect to the other set of record attributes. The other set of record attributes include one or more record attributes different from the one or more strong-identifier-corresponding record attributes. Based on the number of matches in the first set of matches, stopping of the processing of the other collection portion with respect to predicting healthcare record matches is caused.

Another aspect of the disclosure relates to a method for facilitating computer-assisted linkage of healthcare records using strong identifiers. The method comprises processing, using a set of record attributes corresponding to strong identifiers, a first portion of a collection of healthcare records of individuals to predict which healthcare records of the first collection portion have matching values with respect to the set of record attributes. The strong-identifier-corresponding set of record attributes includes one or more record attributes corresponding to strong identifiers. The prediction indicates a first set of matches between healthcare records of the first collection portion. A number of matches in the first set of matches is determined. Using another set of record attributes, processing is performed on at least one other portion of the collection of healthcare records of individuals. This is done to predict which healthcare records of the other collection portion have matching values with respect to the other set of record attributes. The other set of record attributes include one or more record attributes different from the one or more strong-identifier-corresponding record attributes. Based on the number of matches in the first set of matches, stopping of the processing of the other collection portion with respect to predicting healthcare record matches is caused.

Yet another aspect of the disclosure relates to a system configured for facilitating computer-assisted linkage of healthcare records using strong identifiers. The system comprises means for processing, using a set of record attributes corresponding to strong identifiers, a first portion of a collection of healthcare records of individuals to predict which healthcare records of the first collection portion have matching values with respect to the set of record attributes. The strong-identifier-corresponding set of record attributes includes one or more record attributes corresponding to strong identifiers. The prediction indicates a first set of matches between healthcare records of the first collection portion. A number of matches in the first set of matches is determined. Using another set of record attributes, processing is performed on at least one other portion of the collection of healthcare records of individuals. This is done to predict which healthcare records of the other collection portion have matching values with respect to the other set of record attributes. The other set of record attributes include one or more record attributes different from the one or more strong-identifier-corresponding record attributes. Based on the number of matches in the first set of matches, stopping of the processing of the other collection portion with respect to predicting healthcare record matches is caused.

These and other features and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 illustrates a system configured for facilitating computer-assisted linkage of healthcare records, in accordance with one or more embodiments;

FIG. 2 is a schematic diagram of the segmentation of databases, in accordance with one or more embodiments;

FIG. 3 illustrates the classification of hidden matches in tabular format, in accordance with one or more embodiments;

FIG. 4 illustrates the use of a classification algorithm to predict matches, in accordance with one or more embodiments.

FIG. 5 illustrates the use of a distance check, in accordance with one or more embodiments.

FIG. 6 illustrates an example of equations used for a k-cardinality assignment problem, in accordance with one or more embodiments.

FIG. 7 illustrates one method for facilitating computer-assisted linkage of healthcare records, in accordance with one or more embodiments.

FIG. 8 illustrates one method for facilitating computer-assisted linkage of healthcare records, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

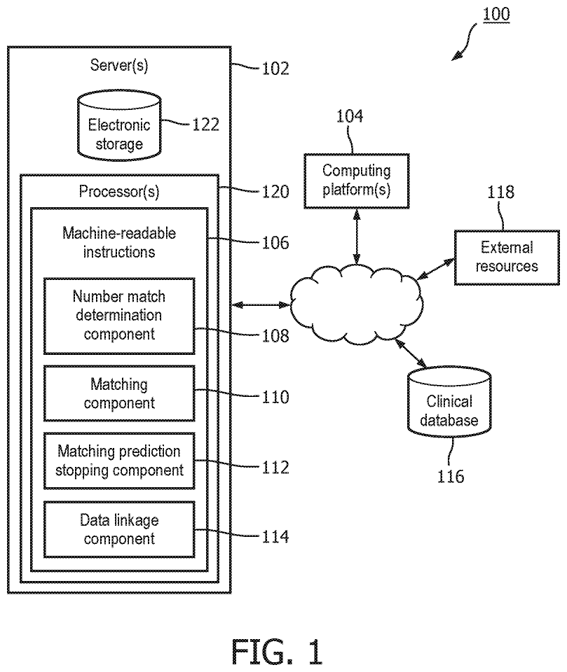

FIG. 1 illustrates a system 100 configured for facilitating computer-assisted linkage of healthcare records, in accordance with one or more embodiments. In some embodiments, system 100 may include one or more servers 102. The server(s) 102 may be configured to communicate with one or more computing platforms 104 according to a client/server architecture, a peer-to-peer architecture, and/or other architectures. The users may access system 100 via computing platform(s) 104. The server(s) 102 may be configured to execute machine-readable instructions 106. The machine-readable instructions 106 may include one or more of a number match determination component 108, a matching component 110, a matching prediction stopping component 112, a data linkage component 114, and/or other machine-readable instruction components.

As mentioned herein, typical data linkage systems waste significant amounts of computational resources continuing to process a collection of records for matches, for example, when few undetermined matches (relative to the amount of matches already found) remain. Although an arbitrary predefined threshold may be strictly enforced to stop processing to limit computational resource waste, the strict use of an arbitrary predefined threshold often results in insufficient matches (e.g., when the same predefined threshold is strictly used on different collections of data). Specifically, for example, while the strict use of a particular predefined threshold for processing a first collection of records may produce sufficiently complete matching, it is very likely that processing of another collection of records using the same predefined threshold will produce insufficiently complete matching, where the other record collection has record inconsistencies different from those in the first record collection, record attributes different from those in the first record collection, or other differences. Additionally, or alternatively, many data linkage systems are configured to determine matches between anonymized data records and, thus, do not rely on personally identifiable information or other strong identifiers for matching and/or linking of data records. Thus, such data linkage systems are not optimized to process collections of non-anonymized data records or other data records with strong identifiers.

In some cases, record matching and/or linking may be performed on a collection of records that include personally identifiable information, such as social security numbers, phone numbers, names, home addresses, etc., or other strong identifiers. Strong identifiers can be used on their own or with other information to identify, contact, or locate an individual, or to identify an individual in context (e.g., in a database of a medical facility). The existence of strong identifiers may, for example, enable a considerable portion of the records to be matched. In some embodiments, “considerable portion” refers to at least 80 percent. In some embodiments, “considerable portion” refers to at least 50 percent. However, corrupt and missing identifiers inevitably lead to a potential large amount of hidden matches left in the database. Data linkage without considering corrupt and missing strong identifiers may result in insufficient linkage, leaving a potentially large amount of hidden matches in the databases. By identifying corrupt identifiers and considering records with missing identifiers, the hidden matches may be uncovered. In some embodiments, other record attributes (e.g., record attributes that do not correspond to strong identifiers) may be used additionally or alternatively to the use of record attributes corresponding to strong identifiers. As an example, these other record attributes may include the patient demographics, acuities, lengths of stay (e.g., at a hospital), or other attributes. In some embodiments, healthcare records may include a plurality of record attributes (e.g., categories of information such as social security number, name, address, date of birth, doctor's name, treating facility, treatment description, treatment date, etc.) and corresponding values for the attributes (e.g., a social security number of 123-45-6789, a name of John P. Doe, 321 Main St., Jan. 1, 1960, etc.). In some embodiments, corresponding attributes and values are attribute-value pairs. In some embodiments, the attribute-value pairs may be a name-value pair, key-value pair, field-value pair, and the like.

In some embodiments, number match determination component 108 is configured for processing, using a set of record attributes corresponding to strong identifiers, a first portion of a collection of healthcare records of individuals to predict which healthcare records of the first collection portion have matching values with respect to the set of record attributes. As an example, two healthcare records may be determined to have matching values with respect to a record attribute responsive to a determination that both records have the same value for that record attribute (e.g., both records include the attribute-value pair of “SSN=212-12-1234”). As another example, two healthcare records may be determined to have matching values with respect to a record attribute responsive to a determination that both records have similar values for that record attribute, where the similar values satisfy a similarity threshold (e.g., a particular edit distance from one another, a particular Euclidean distance from one another, etc.). As a further example, responsive to two records have matching values with respect to a certain record attribute (e.g., SSN) or combination of record attributes (e.g., first name and last name), it may be determined that both records correspond to the same individual. As an example, the strong-identifier-corresponding set of record attributes may include one or more record attributes corresponding to strong identifiers. The prediction may indicate a first set of matches between healthcare records of the first collection portion. In some embodiments, number match determination component 108 is configured for determining a number of matches in the first set of matches. As an example, the number of matches may include a percentage of matches in the first set of matches, a quantity of matches in the first set of matches, etc. In some embodiments, data linkage component 114 is configured for linking healthcare records of the first collection portion based on the first set of matches.

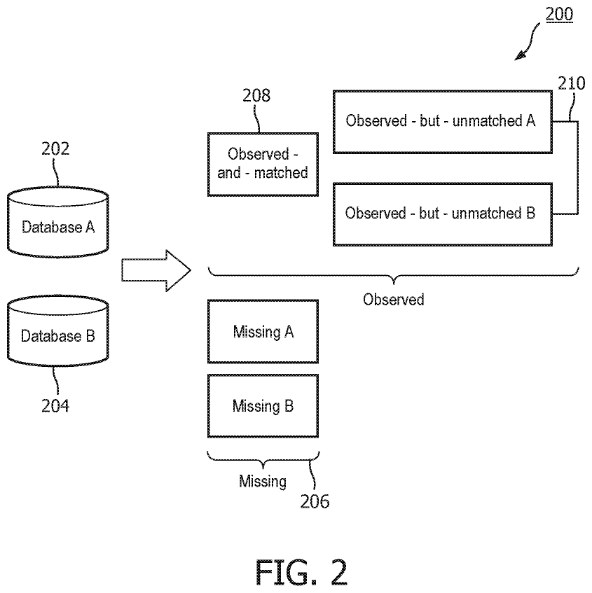

In one use case, with respect to FIG. 2, a collection of healthcare records originating from database A 202 and the database B 204 may be processed. During the processing, the collection of healthcare records may be segmented into various portions according to a strong identifier (e.g., social security number or other strong identifier). The segmented portions may, for instance, include a missing portion 206, an observed-and-matched portion 208, and an observed-but-unmatched portion 210. As an example, missing portion 206 may include records that are each lacking social security numbers (e.g., records having no record attributes corresponding to a social security number, records for which a social security number was never entered, etc.). Observed-and-matched portion 208 may include records that have social security numbers and have been matched with at least one other record (or at least a predefined threshold number of records) in the record collection based on the respective matching records having the same social security number. Observed-and-unmatched portion 210 may include records that have social security numbers but have not yet been matched with at least one other record (or at least a predefined threshold number of records) in the record collection (e.g., records having a social security number that did not match any social security number of other records in the record collection or did not match the threshold number of records with respect to their social security numbers). For observed-and-matched portion 208, it may be assumed that identifier corruptions do not result in false positives (i.e., different individuals incorrectly designated as having the same social security number due to data entry errors). While matches have been determined for observed-and-matched portion 208, there may be hidden matches in missing portion 206 and observed-but-unmatched portion 210.

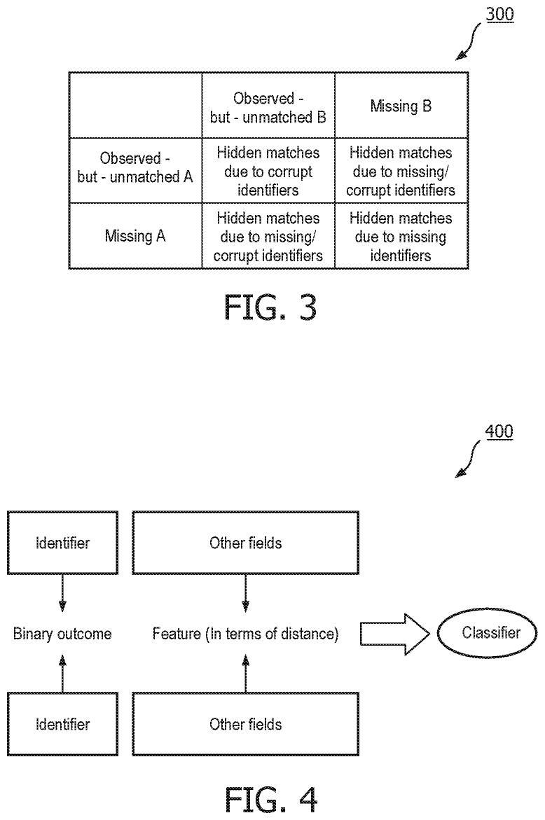

In a further use case, table 300 of FIG. 3 shows four types of hidden matches in terms of from where the match pair came. In particular, if one were to only consider the observed-but-unmatched portion (indicated in the upper left of the four cells of table 300), then hidden matches therein may be due to identifier corruptions, for instance, because a given strong identifier has been observed in each of those records (i.e., the value field for the strong identifier attribute is filled in and is not missing).

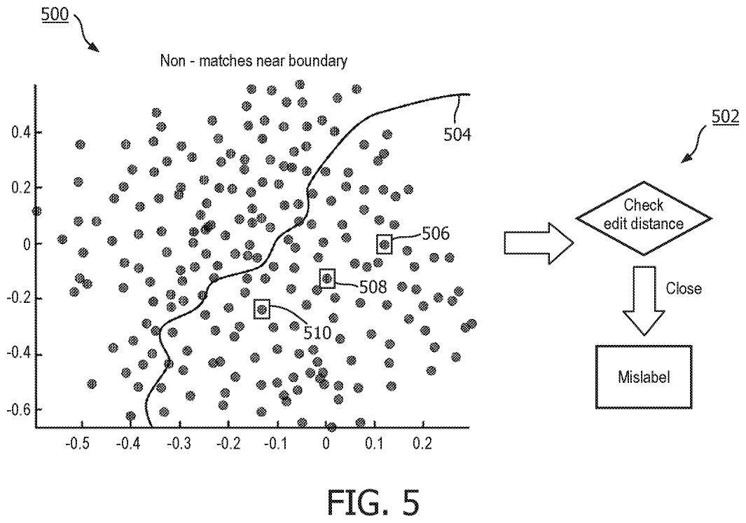

With respect to FIGS. 4 and 5, for example, system 100 may process the observed-but-unmatched portion. In one scenario, if social security number is the strong identifier used to determine the matching records of the observed-and-matched portion, matching score engine 400 may perform an edit distance technique with respect to social security numbers of records of the observed-but-unmatched portion to determine additional record matches. In another scenario, values for other record attributes of database A 202 and database B 204 can be used to assist in identifying matches between records with mislabelled social security numbers. As an example, matching score engine 400 may use the determination of whether observed social security numbers of a pair of records were a match as the binary outcome, and may use the similarities or differences between values of the other record attributes, such as the patient demographics, acuities, lengths of stay, etc., as additional information to determine whether a match between a pair of records exist (e.g., to confirm or override the binary outcome derived from use of social security number as the strong identifier). There are different choices of measures, depending on the type of the data field. For example, Euclidean distance may be more appropriate for certain numerical fields such as, for example, an individual's age and length of stay in a medical facility. As another example, edit distance might be used for certain string fields. In some circumstances, a machine learning classification model may be used to perform record matching predictions, and can return a score related to the likelihood of being a match for each pair. Examples of classification algorithms include one or more of logistic regression, support vector machine, random forest, and/or other algorithms.

As discussed, in some cases with respect to FIG. 5, a distance check (e.g., edit distance check) may be used to determine record matches. As an example, plot 500 and schematic 502 may relate to the process of identifying hidden matches due to identifier corruptions. In some embodiments, a classification algorithm is used to screen candidate matches, and then a distance check of respective strong identifiers is performed. Mislabelled false negative matching record pairs may have high matching scores. System 100 first identifies the unmatched pairs with high matching scores (or equivalently, pairs near the classification boundary 504 such as dots 506, 508, and 510 with boxes around them in FIG. 5). Also note that some of the dots have vertical cross-hatching, and some of the dots have diagonal cross-hatching in order to differentiate them as being related to different databases. This screening provides a list of possible hidden matches. The strong identifiers of these potential hidden matches are then examined, and if the strong identifiers are sufficiently close (for example, a social security number of 123-45-6789 as compared to a social security number of 123-54-6789), they are declared as hidden matches due to identifier corruptions and relabelled as matches.

In some embodiments, matching component 110 is configured for processing, using another set of record attributes, at least one other portion of the collection of healthcare records of individuals. As an example, this may be done to predict which healthcare records of the other collection portion have matching values with respect to the other set of record attributes. The other set of record attributes may include one or more record attributes different from the strong-identifier-corresponding record attributes (used to process the first collection portion). Data linkage component 114 also links healthcare records of the other collection portion based on a second set of matches derived from the processing of the other collection portion, the second set of matches being derived prior to the stopping of the processing of the other collection portion.

In one use case, for example, a collection of healthcare records may include 1,000,000 or more healthcare records, where the first portion of the collection of healthcare records may include 100,000 records, and another other portion of the collection portion of healthcare records may include 900,000 records. The strong-identifier-corresponding record attributes may be used to process the 100,000 records of the first collection portion, and the record attributes (different from the strong-identifier-corresponding record attributes) may be used to process the 900,000 records of the other collection portion. In some embodiments, the other collection portion may not include the first collection portion (e.g., the other collection portion do not include one or more records of the first collection portion, the records of the first collection portion and the records of the other collection portion are mutually exclusive, etc.). In some embodiments, the other collection portion may include the first collection portion. As an example, the first collection portion may be a subset of the other collection portion.

In some embodiments, matching prediction stopping component 112 is configured for causing, based on the number of matches in the first set of matches (predicted based on the processing of the first collection portion), stopping of the processing of the other collection portion with respect to predicting healthcare record matches. As an example, matching prediction stopping component may determine one or more stopping criteria based on the number of matches in the first set of matches, monitor the processing of the other collection portion based on the stopping criteria, and stop the processing of the other collection portion with respect to predicting healthcare record matches based on the stopping criteria.

In some embodiments, matching prediction stopping component 112 is configured for determining a first threshold as a stopping criterion based on the number of matches (in the first set of matches). Prior to the stopping, matching prediction stopping component 112 may determine whether to continue the processing of the other collection portion based on the first threshold. If, for example, it is determined that a number of matches between healthcare records of the other collection portion has satisfied the first threshold, matching prediction stopping component 112 may cause the stopping of the processing of the other collection portion with respect to predicting healthcare record matches. On the other hand, if it is determined that the number of matches between healthcare records of the other collection portion has not yet satisfied the first threshold, matching prediction stopping component 112 may continue the processing of the other collection portion.

As an example, if a given percentage (e.g., 70%) of the records in the first collection portion were matched, the processing of the other collection portion may be stopped once (i) the number of matches are at least within 10 percentage points of the 70% match prediction (for the first collection portion) (e.g., at least 60%) and (ii) the processing of the other collection portion has been performed for a given amount of time. As another example, the number of matches in the first set of matches may be used to set an absolute stopping point of the processing of the other collection portion. In one scenario, for instance, the processing of the other collection portion may be stopped once the number of matches is more than 10 percentage points of the 70% match prediction (for the first collection portion). In a further scenario, one or more other stopping criteria may be utilized to cause the processing of the other collection portion to be stopped prior to the predetermined absolute stopping point. It is should be noted that these numbers are merely examples, and the use of other numbers (e.g., other percentages, other quantities, etc.) are contemplated.

As a further example, matching prediction stopping component 112 may cause the processing (of the other collection portion) to be stopped responsive to at a time subsequent a determination that that the number of matches (between healthcare records of the other collection portion) has not yet satisfied the first threshold has been reached. In one use case, for instance, the subsequent time may be a time at which the processing of the other collection portion is pre-set to end. In a further use case, matching prediction stopping component 112 may set the subsequent time (as the end time of the processing of the other collection portion) based on the number of matches (in the first set of matches).

In some embodiments, matching prediction stopping component 112 may determine a second threshold as a stopping criterion based on the number of matches (in the first set of matches), where the second threshold is different from the first threshold. As an example, if it is determined that the number of matches between healthcare records of the other collection portion has not yet satisfied the first threshold at a first time (e.g., a time t₁ at which a stopping determination is to be effectuated), matching prediction stopping component 112 may continue the processing of the other collection portion. At a second time (e.g., a time t₂ at which a stopping determination is to be effectuated), matching prediction stopping component 112 may determine whether the number of matches between healthcare records of the other collection portion satisfies the second threshold (e.g., a lower threshold relative to the first threshold). If, at the second time, it is determined that the number of matches between healthcare records of the other collection portion satisfies the second threshold, matching prediction stopping component 112 may cause the stopping of the processing of the other collection portion with respect to predicting healthcare record matches. Otherwise, in some embodiments, the processing of the other collection portion for healthcare record matches may be continued.

In this way, for example, the stopping of the processing of one or more collection portions for healthcare record matches (or other data record matches) based on the number of matches (in the first set of matches) may save computational resources, such as processing resources, memory resources, network bandwidth, etc., while addressing the problems associated with the strict use of the same predefined threshold for different record collections. As an example, the stopping of further prediction of record matches for a record collection (or a portion thereof) may be individualized for the record collection based on the number of matches (in a small subset of the record collection) derived by first processing that small collection subset. As another example, the use of strong identifiers to perform the matching of records in the small collection subset may facilitate a sufficiently accurate count of the number of record matches that exist in the small collection subset and, thus, when that number of record matches is used to determine one or more stopping criteria on which subsequent processing of one or more other subsets of the record collection is to be based, the resulting matches derived from such processing may be sufficiently complete (e.g., a low number of false positives and/or false positives, a high number of true positives and/or true negatives, etc.).

In some embodiments, a total number of matches with respect to at least a portion of may be determined as follows, for example, with respect to the examples described herein having one or more observed-and-matched portions and observed-but-unmatched portions. Once the hidden matches due to identifier corruptions has been recovered, system 100 has obtained all the matches for the observed portions of database A 202 database B 204. A matching rate can be defined as follows.

$$α = \frac{\# \{ matches of observed A and observed B \}}{\# \{ observed A \} \times \# \{ observed B \}}$$

This matching rate measures the naturally occurring rate of matches between database A 202 and database B 204. Generalizing this occurring rate to the whole database, system 100 estimates the number of matches k across all portions of database A 202 and database B 204 by the following equation.

k=α×#{A}×#{B}

This number k gives an idea of how many more matches could be found if all identifiers were observed perfectly.

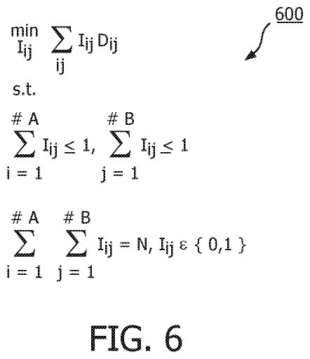

FIG. 6 illustrates an example of equations used for solving a k-cardinality assignment problem, in accordance with one or more embodiments. With the total number of matches k having been determined, the data linkage problem may be reformulated as a classic combinatorial optimization problem (i.e., a k-cardinality assignment problem). k links are identified between records in database A 202 and database B 204 that gives the minimum distance, where the distance is defined over the fields of the records. Distance measures used in the previous steps could be used here. Those matches that have already been identified can be removed first. The mathematical description is given in FIG. 6. Dij denotes the distance between record i in database A 202 and record j in database B 204. Iij denotes an indicator variable with the number 1 indicating record i in database A 202 and record j in database B 204 are matched. The number 0 indicates otherwise. In some embodiments, the constraints are such that a given record cannot be matched to more than one record in the other database, and there are k matches in total. In other embodiments, a given record can be matched to two or more records in the other database. The # symbol indicates the size of the set under consideration, such as the number of records in database A 202 for example.

In some embodiments, system 100 comprises one or more databases (e.g., clinical database 116), one or more computing platforms 104, one or more processors 120, electronic storage 122, external resources 118, and/or other components.

Clinical database(s) 116 are configured to electronically store healthcare records of individuals and/or other information. As previously mentioned, the healthcare records may include a plurality of record attributes and corresponding values for the attributes.

In some embodiments, the databases (e.g., clinical database 116) are associated with one or more entities such as medical facilities (e.g., hospitals, doctor's offices, etc.), healthcare management providers (e.g., a veteran's affairs medical system, a ministry of health), health insurance providers, and/or other entities. Databases 12 comprise electronic storage media that electronically stores information. In some embodiments, databases 116 are and/or are included in computers, servers, and/or other data storage systems associated with the one or more entities. The electronic storage media of databases 116 may comprise system storage that is provided integrally (i.e., substantially non-removable) with such systems. Databases 116 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Databases 116 are configured to communicate with computing platforms 104, processor 120, electronic storage 122, external resources 118, and/or other components of system 100 such that the information stored by databases 116 may be accessed (e.g., as described herein) by other components of system 100 and/or other systems. It should be noted that use of the term “databases” is not intended to be limiting. A database may be any electronic storage system that stores healthcare records and allows system 100 to function as described herein.

Computing platforms 104 are configured to provide an interface between users and system 100. In some embodiments, computing platforms 104 are associated with databases 116, processor 120 and/or a server that includes processor 120, a healthcare provider, individual users associated with the healthcare provider, service providers (e.g., consultants) to the healthcare provider, individual users of system 100, and/or other users and/or entities. Computing platforms 104 are configured to provide information to and/or receive information from such users and/or entities. Computing platforms 104 include a user interface and/or other components. The user interface may be and/or include a graphical user interface configured to present views and/or fields configured to receive entry and/or selection of healthcare records and/or information associated with healthcare records, present information related to matched healthcare records (e.g., matching probabilities, F-scores, record attributes), and/or provide and/or receive other information. In some embodiments, the user interface includes a plurality of separate interfaces associated with a plurality of computing platforms 104, processors 120, and/or other components of system 100, for example.

In some embodiments, one or more computing platforms 104 are configured to provide a user interface, processing capabilities, databases, and/or electronic storage to system 100. As such, computing platforms 104 may include processors 120, electronic storage 122, external resources 118, and/or other components of system 100. In some embodiments, computing platforms 104 are connected to a network (e.g., the internet). In some embodiments, computing platforms 104 do not include processor 120, electronic storage 122, external resources 118, and/or other components of system 100, but instead communicate with these components via the network. The connection to the network may be wireless or wired. For example, processor 120 may be located in a remote server and may wirelessly receive healthcare records for matching from one or more healthcare providers. In some embodiments, computing platforms 104 are laptops, desktop computers, smartphones, tablet computers, and/or other computing devices.

Examples of interface devices suitable for inclusion in the user interface include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that computing platforms 104 include a removable storage interface. In this example, information may be loaded into computing platforms 104 from removable storage (e.g., a smart card, a flash drive, a removable disk) that enables users to customize the implementation of computing platforms 104. Other exemplary input devices and techniques adapted for use with computing platforms 104 and/or the user interface include, but are not limited to, an RS-232 port, RF link, an IR link, a modem (telephone, cable, etc.) and/or other devices.

As shown in FIG. 1, processor 120 is configured via machine-readable instructions to execute one or more computer program components. Processor 120 may be configured to execute components 108, 110, 112, and/or 114 by software; hardware; firmware; some combination of software, hardware, and/or firmware; and/or other mechanisms for configuring processing capabilities on processor 120.

It should be appreciated that although components 108, 110, 112, and 114 are illustrated in FIG. 1 as being co-located within a single processing unit, in embodiments in which processor 120 comprises multiple processing units, one or more of components 108, 110, 112, and/or 114 may be located remotely from the other components. The description of the functionality provided by the different components 108, 110, 112, and/or 114 described below is for illustrative purposes, and is not intended to be limiting, as any of components 108, 110, 112, and/or 114 may provide more or less functionality than is described. For example, one or more of components 108, 110, 112, and/or 114 may be eliminated, and some or all of its functionality may be provided by other components 108, 110, 112, and/or 114. As another example, processor 120 may be configured to execute one or more additional components that may perform some or all of the functionality attributed below to one of components 108, 110, 112, and/or 114.

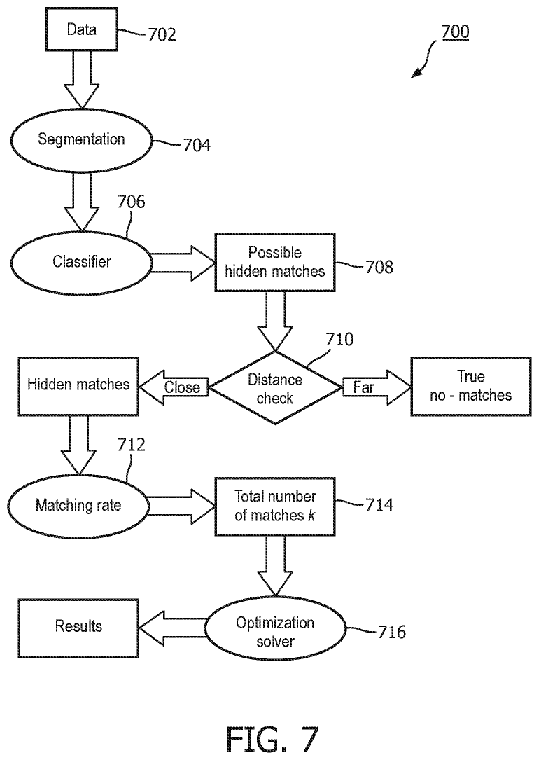

FIG. 7 illustrates one method for facilitating computer-assisted linkage of healthcare records using strong identifiers, in accordance with one or more embodiments. The operations of method 700 presented below are intended to be illustrative. In some embodiments, method 700 may be accomplished with one or more additional operations not described, and/or without one or more of the operations discussed. Additionally, the order in which the operations of method 700 are illustrated in FIG. 7 and described below is not intended to be limiting.

In some embodiments, one or more operations of method 700 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 700 in response to instructions stored electronically on an electronic storage medium. The one or more processing devices may include one or more devices configured through hardware, firmware, and/or software to be specifically designed for execution of one or more of the operations of method 700.

At an operation 702, data from database A 202 and database B 204 is received by server(s) 102. Operation 702 may be performed by one or more hardware processors 120 configured to execute a machine-readable instruction component that is the same as or similar to one or more of components 108, 110, 112, and/or 114 (as described in connection with FIG. 1), in accordance with one or more implementations.

At an operation 704, the data from database A 202 and database B 204 is segmented into three portions according to the strong identifier, as discussed herein. Operation 704 may be performed by one or more hardware processors 120 configured to execute a machine-readable instruction component that is the same as or similar to one or more of components 108, 110, 112, and/or 114 (as described in connection with FIG. 1), in accordance with one or more implementations.

At an operation 706, the data is classified by a classification algorithm to predict matches based on information in various fields of database A 202 and database B 204. Operation 706 may be performed by one or more hardware processors 120 configured to execute a machine-readable instruction component that is the same as or similar to one or more of components 108, 110, 112, and/or 114 (as described in connection with FIG. 1), in accordance with one or more implementations.

At an operation 708, possible hidden matches are identified. As an example, these hidden matches may be due to corrupt identifiers. Operation 708 may be performed by one or more hardware processors 120 configured to execute a machine-readable instruction component that is the same as or similar to one or more of components 108, 110, 112, and/or 114 (as described in connection with FIG. 1), in accordance with one or more implementations.

At an operation 710, a check of edit distance is performed. If the edit distance between the strong identifiers is far between potential matches, then the potential matches are labelled as true non-matches. If the distance between the strong identifiers is close between potential matches, then the potential matches are labelled as hidden matches. Operation 710 may be performed by one or more hardware processors 120 configured to execute a machine-readable instruction component that is the same as or similar to one or more of components 108, 110, 112, and/or 114 (as described in connection with FIG. 1), in accordance with one or more implementations.

At an operation 712, a matching rate is determined. A quantity is determined that produces the naturally occurring rate of matches between database A 202 and database B 204. Operation 712 may be performed by one or more hardware processors 120 configured to execute a machine-readable instruction component that is the same as or similar to one or more of components 108, 110, 112, and/or 114 (as described in connection with FIG. 1), in accordance with one or more implementations.

At an operation 714, a number k is calculated. This number gives an estimation of how many more matches could be found if all identifiers were observed perfectly. In other words, the total number of matches is estimated. Operation 714 may be performed by one or more hardware processors 120 configured to execute a machine-readable instruction component that is the same as or similar to one or more of components 108, 110, 112, and/or 114 (as described in connection with FIG. 1), in accordance with one or more implementations.

At an operation 716, with the total number of matches k having been determined, the data linkage problem is reformulated as a classic combinatorial optimization problem (i.e., a k-cardinality assignment problem). k links are identified between records in database A 202 and database B 204 that give the minimum distance, where the distance is defined over the fields of the records. Operation 716 may be performed by one or more hardware processors 120 configured to execute a machine-readable instruction component that is the same as or similar to one or more of components 108, 110, 112, and/or 114 (as described in connection with FIG. 1), in accordance with one or more implementations.

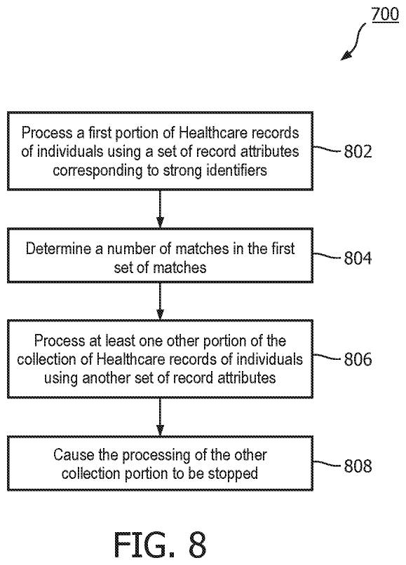

FIG. 8 illustrates one method 800 for facilitating computer-assisted linkage of healthcare records using strong identifiers, in accordance with one or more embodiments. The operations of method 800 presented below are intended to be illustrative. In some embodiments, method 800 may be accomplished with one or more additional operations not described, and/or without one or more of the operations discussed. Additionally, the order in which the operations of method 800 are illustrated in FIG. 8 and described below is not intended to be limiting.

In some embodiments, one or more operations of method 800 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 800 in response to instructions stored electronically on an electronic storage medium. The one or more processing devices may include one or more devices configured through hardware, firmware, and/or software to be specifically designed for execution of one or more of the operations of method 800.

At an operation 802, a first portion of a collection of healthcare records of individuals may be processed using a set of record attributes corresponding to strong identifiers. As an example, this may be done to predict which healthcare records of the first collection portion have matching values with respect to the set of record attributes. The strong-identifier-corresponding set of record attributes may include one or more record attributes corresponding to strong identifiers. The prediction may indicate a first set of matches between healthcare records of the first collection portion. Operation 802 may be performed by one or more hardware processors 120 configured to execute a machine-readable instruction component that is the same as or similar to one or more of components 108, 110, 112, and/or 114 (as described in connection with FIG. 1), in accordance with one or more implementations.

At an operation 804, a number of matches in the first set of matches is determined. Operation 804 may be performed by one or more hardware processors 120 configured to execute a machine-readable instruction component that is the same as or similar to one or more of components 108, 110, 112, and/or 114 (as described in connection with FIG. 1), in accordance with one or more implementations.

At an operation 806, at least one other portion of the collection of healthcare records of individuals may be processed using another set of record attributes. As an example, this may be done to predict which healthcare records of the other collection portion have matching values with respect to the other set of record attributes. The other set of record attributes may include one or more record attributes different from the one or more strong-identifier-corresponding record attributes. Operation 806 may be performed by one or more hardware processors 120 configured to execute a machine-readable instruction component that is the same as or similar to one or more of components 108, 110, 112, and/or 114 (as described in connection with FIG. 1), in accordance with one or more implementations.

At an operation 808, the processing of the other collection portion (with respect to predicting healthcare record matches) is caused to be stopped based on the number of matches in the first set of matches. Operation 808 may be performed by one or more hardware processors 120 configured to execute a machine-readable instruction component that is the same as or similar to one or more of components 108, 110, 112, and/or 114 (as described in connection with FIG. 1), in accordance with one or more implementations.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A system configured for facilitating computer-assisted linkage of healthcare records using strong identifiers, the system comprising:
one or more hardware processors configured by machine-readable instructions to:
process, using a set of record attributes corresponding to strong identifiers, a first portion of a collection of healthcare records of individuals to predict which healthcare records of the first collection portion have matching values with respect to the set of record attributes, the strong-identifier-corresponding set of record attributes including one or more record attributes corresponding to strong identifiers, and the prediction indicating a first set of matches between healthcare records of the first collection portion;
determine a number of matches in the first set of matches;
process, using another set of record attributes, at least one other portion of the collection of healthcare records of individuals to predict which healthcare records of the other collection portion have matching values with respect to the other set of record attributes, the other set of record attributes includes one or more record attributes different from the one or more strong-identifier-corresponding record attributes; and
cause, based on the number of matches in the first set of matches, stopping of the processing of the other collection portion with respect to predicting healthcare record matches.

2. The system of claim 1, wherein strong identifiers include personally identifiable information, and wherein the strong-identifier-corresponding set of record attributes includes one or more record attributes corresponding to personally identifiable information.

3. The system of claim 1, wherein the one or more hardware processors are configured to:
link healthcare records of the first collection portion based on the first set of matches; and
link healthcare records of the other collection portion based on a second set of matches derived from the processing of the other collection portion, the second set of matches being derived prior to the stopping of the processing of the other collection portion.

4. The system of claim 1, wherein the one or more hardware processors are configured to:
determine a first threshold based on the number of matches;
determine, prior to the stopping, whether to continue the processing of the other collection portion based on the first threshold; and
continue the processing of the other collection portion responsive to a determination that a number of matches between healthcare records of the other collection portion has not yet satisfied the first threshold.

5. The system of claim 1, wherein the other collection portion does not include the first collection portion.

6. The system of claim 1, wherein the other collection portion includes at least some of the first collection portion.

7. The system of claim 1, wherein the number of matches in the first set of matches is a percentage of records that were predicted to have matched during the processing of the first collection portion, and wherein the one or more hardware processors are configured to cause the stopping of the processing of the other collection portion by causing, based on the percentage of matched records, the stopping of the processing of the other collection portion with respect to predicting healthcare record matches.

8. The system of claim 1, wherein the number of matches in the first set of matches is a quantity of records that were predicted to have matched during the processing of the first collection portion, and wherein the one or more hardware processors are configured to cause the stopping of the processing of the other collection portion by causing, based on the quantity of matched records, the stopping of the processing of the other collection portion with respect to predicting healthcare record matches.

9. A method for facilitating computer-assisted linkage of healthcare records using strong identifiers, the method comprising:
processing, using a set of record attributes corresponding to strong identifiers, a first portion of a collection of healthcare records of individuals to predict which healthcare records of the first collection portion have matching values with respect to the set of record attributes, the strong-identifier-corresponding set of record attributes including one or more record attributes corresponding to strong identifiers, and the prediction indicating a first set of matches between healthcare records of the first collection portion;
determining a number of matches in the first set of matches;
processing, using another set of record attributes, at least one other portion of the collection of healthcare records of individuals to predict which healthcare records of the other collection portion have matching values with respect to the other set of record attributes, the other set of record attributes includes one or more record attributes different from the one or more strong-identifier-corresponding record attributes; and
causing, based on the number of matches in the first set of matches,
stopping of the processing of the other collection portion with respect to predicting healthcare record matches.

10. The method of claim 9, wherein strong identifiers include personally identifiable information, and wherein the strong-identifier-corresponding set of record attributes includes one or more record attributes corresponding to personally identifiable information.

11. The method of claim 9, further comprising:
linking healthcare records of the first collection portion based on the first set of matches; and
linking healthcare records of the other collection portion based on a second set of matches derived from the processing of the other collection portion, the second set of matches being derived prior to the stopping of the processing of the other collection portion.

12. The method of claim 9, further comprising:
determining a first threshold based on the number of matches;
determining, prior to the stopping, whether to continue the processing of the other collection portion based on the first threshold; and
continuing the processing of the other collection portion responsive to a determination that a number of matches between healthcare records of the other collection portion has not yet satisfied the first threshold.

13. The method of claim 9, wherein the other collection portion does not include the first collection portion.

14. The system of claim 9, wherein the other collection portion includes at least some of the first collection portion.

15. A system for facilitating computer-assisted linkage of healthcare records using strong identifiers, the system comprising:
means for processing, using a set of record attributes corresponding to strong identifiers, a first portion of a collection of healthcare records of individuals to predict which healthcare records of the first collection portion have matching values with respect to the set of record attributes, the strong-identifier-corresponding set of record attributes including one or more record attributes corresponding to strong identifiers, and the prediction indicating a first set of matches between healthcare records of the first collection portion;
means for determining a number of matches in the first set of matches;
means for processing, using another set of record attributes, at least one other portion of the collection of healthcare records of individuals to predict which healthcare records of the other collection portion have matching values with respect to the other set of record attributes, the other set of record attributes includes one or more record attributes different from the one or more strong-identifier-corresponding record attributes; and
means for causing, based on the number of matches in the first set of matches, stopping of the processing of the other collection portion with respect to predicting healthcare record matches.

16. The system of claim 15, wherein strong identifiers include personally identifiable information, and wherein the strong-identifier-corresponding set of record attributes includes one or more record attributes corresponding to personally identifiable information.

17. The system of claim 15, further comprising:
means for linking healthcare records of the first collection portion based on the first set of matches; and
means for linking healthcare records of the other collection portion based on a second set of matches derived from the processing of the other collection portion, the second set of matches being derived prior to the stopping of the processing of the other collection portion.

18. The system of claim 15, further comprising:
means for determining a first threshold based on the number of matches;
means for determining, prior to the stopping, whether to continue the processing of the other collection portion based on the first threshold; and
means for continuing the processing of the other collection portion responsive to a determination that a number of matches between healthcare records of the other collection portion has not yet satisfied the first threshold.

19. The system of claim 15, wherein the other collection portion does not include the first collection portion.

20. The system of claim 15, wherein the other collection portion includes at least some of the first collection portion.
